(19) United States
(12) Patent Application Publication
(10) Pub. No.: US 2009/0326543 A1
(43) Pub. Date: Dec. 31, 2009

(54) SPINE SURGERY METHOD AND EXTRACTOR

(76) Inventor: Henry F. Fabian, JR., Steamboat Springs, CO (US)

Correspondence Address: Emerson, Thomson & Bennett, LLC, 777 W. Market Street, Akron, OH, 44303, US

(21) Appl. No.: 12/164,561

(22) Filed: Jun. 30, 2008

(51) Int. Cl. A61B 17/58; A61B 19/00

(52) U.S. Cl. 606/99; 128/898

(57) ABSTRACT

A surgical extractor for use in extracting an implant from a vertebral space may include: (1) a handle, having an opening, for use by a surgeon in maneuvering the surgical extractor; and (2) an implant gripping mechanism having: (a) a first grip member with one end attached to the handle and a second end with a first contact surface; and, (b) a second grip member with one end threaded in engagement to the opening in the handle and a second end with a second contact surface. The handle can be rotated with respect to the second grip member to cause the first and second contact surfaces to move relatively toward each other to grip the implant so that the implant can be extracted from the vertebral space.

### BACKGROUND OF THE INVENTION

A. Field of Invention

This invention pertains to the art of methods and apparatuses regarding spine surgery and more specifically relates to surgical procedures and an extractor used to remove an implant from a vertebral space, and perhaps to un-deploy the implant within a vertebral space.

B. Description of the Related Art

The volume of spinal surgeries to treat degenerative disc and facet disease has steadily increased over the past decade, fueled by population demographics and advancements in diagnostic and instrumentation adjuncts. Improvements in intraoperative radiological imaging and surgical technique have generated a great deal of interest in applying minimally invasive surgical (MIS) techniques to spinal applications. As in other surgical subspecialties, it is hoped such minimally invasive techniques applied to spinal surgery will result in less soft tissue trauma, less operative blood loss, reduced operative time, faster recovery periods and lower costs.

Known spinal surgical techniques, though generally working well for their intended purposes, have been adopted from traditional open surgical (non-MIS) techniques. As a result, known spinal surgical methods, instrumentation and interbody implants have limitations. One limitation is that the physical components are relatively large and bulky. This reduces surgeon visualization of the surgical site. Another limitation of known spinal surgical methods is that known surgical tools and implants are cumbersome and difficult to maneuver within the limited surgical space available. The limitations of current instrumentation in MIS spine surgery are noted particularly with regards to interbody fusion surgery.

The present invention provides methods and apparatuses for overcoming these limitations by providing a surgical extractor that allows for minimally invasive spinal surgery and that provides for precise movement, placement and undeployment of an implant within the vertebral space.

### SUMMARY OF THE INVENTION

According to one embodiment of this invention, a surgical extractor for use in extracting an implant from a vertebral space, may include: (1) a first handle for use by a surgeon in maneuvering the surgical extractor, the first handle having an opening; and, (2) an implant gripping mechanism comprising: (A) a first grip member having a first end attached to the first handle and a second end comprising a first contact surface; and, (B) a second grip member having a first end threadingly received within the opening in the first handle and a second end comprising a second contact surface. The first handle can be rotated in a first direction with respect to the second grip member to cause the first and second contact surfaces to move relatively toward each other to grip the implant. The first handle can also be rotated in a second direction with respect to the second grip member to cause the first and second contact surfaces to move relatively away from each other to release the implant.

According to another embodiment of this invention, the first and second contact surfaces are shaped to match the surfaces of the implant which they contact.

According to another embodiment of this invention, the extractor also has a second handle for use by a surgeon in maneuvering the surgical extractor.

According to yet another embodiment of this invention, a surgical system may include: (I) an implant comprising: (A) a first portion having a first engagement surface; (B) a second portion having a second engagement surface; and, (C) the implant may be deployed within a vertebral space by moving the second portion with respect to the first portion in a first direction; and, (II) a surgical extractor for use in un-deploying the implant within the vertebral space and for use in extracting the implant from the vertebral space, the surgical extractor comprising: (A) a first handle for use by a surgeon in maneuvering the surgical extractor, the first handle having an opening; (B) an implant gripping mechanism comprising: (1) a first grip member having a first end attached to the first handle and a second end comprising a first contact surface; and, (2) a second grip member having a first end threadingly received within the opening in the first handle and a second end comprising a second contact surface. The first handle may be rotated in a first direction with respect to the second grip member to: (1) cause the first contact surface to contact the first engagement surface; (2) cause the second contact surface to contact the second engagement surface; (3) cause the implant to be un-deployed within the vertebral space by moving the second portion with respect to the first portion in a second direction that is substantially different than the first direction; and, (4) grip the implant for removal from the vertebral space.

According to another embodiment of this invention, the first engagement surface is on a first implant post; and, the second engagement surface is on a second implant post.

According to still another embodiment of this invention, a method comprising the steps of: (A) providing an implant positioned within a vertebral space, the implant having first and second engagement surfaces; (B) providing a surgical extractor comprising: (1) a first handle for use by a surgeon in maneuvering the surgical extractor, the first handle having an opening; (2) an implant gripping mechanism comprising: (a) a first grip member having a first end attached to the first handle and a second end comprising a first contact surface; and, (b) a second grip member having a first end threadingly received within the opening in the first handle and a second end comprising a second contact surface; (C) providing access to the vertebral space; (D) rotating the first handle with respect to the second grip member to cause the first and second contact surfaces to move relatively toward each other; (E) continue rotating the first handle with respect to the second grip member to cause the first and second contact surfaces to engage the first and second engagement surfaces, respectively, to grip the implant; and, (F) moving the surgical extractor and thereby the implant away from the vertebral space.

One advantage of this invention is that the inventive surgical extractor permits an implant to be relatively easily removed from a vertebral space.

Another advantage of this invention is that the implant may be relatively easily and securely attached to the extractor and then detached from the extractor.

Another advantage of this invention is that, in one embodiment, the extractor can be used to un-deploy the implant.

Yet another advantage of this invention is that the surgical extractor allows for minimally invasive application via either an anterior, anterolateral, posterior or posterolateral approach, with the latter approach possible via either a transforaminal or extraforaminal approach.

Still other benefits and advantages of the invention will become apparent to those skilled in the art to which it pertains upon a reading and understanding of the following detailed specification.

### BRIEF DESCRIPTION OF THE DRAWINGS

The invention may take physical form in certain parts and arrangement of parts, embodiments of which will be described in detail in this specification and illustrated in the accompanying drawings which form a part hereof and wherein:

FIG. 1 is a side perspective view of a spinal segment showing a vertebral space defined by the intradiscal space usually occupied by a disc between two adjacent vertebral bodies.

FIG. 2 is a side perspective view of a spinal segment showing a vertebral space defined by the space usually occupied by a vertebral body and its two adjacent discs.

FIG. 3 is a top view of an extractor according to one embodiment of this invention.

FIG. 4 is a top view of an extractor similar to that shown in FIG. 3 but showing some components in a see-through condition.

FIG. 5 is a top view of an extractor shown in FIG. 4 but shown without the implant.

FIG. 6 is a perspective proximal end view of a handle according to one embodiment of this invention.

FIG. 7 is a perspective distal end view of the handle shown in FIG. 6.

FIG. 8 is a side sectional view of the handle shown in FIG. 6.

FIG. 9 is a perspective proximal end view of a first grip member according to one embodiment of this invention.

FIG. 10 is a perspective distal end view of the first grip member shown in FIG. 9.

FIG. 11 is a side view of a retaining member according to one embodiment of this invention.

FIG. 12 is an end perspective view of the retaining member shown in FIG. 11.

FIG. 13 is a side view of a second grip member according to one embodiment of this invention.

FIG. 14 is a close-up side view of the distal end of the second grip member shown in FIG. 13.

FIG. 15 is a close-up proximal end view of the proximal end of the extractor showing some components in a see-through condition.

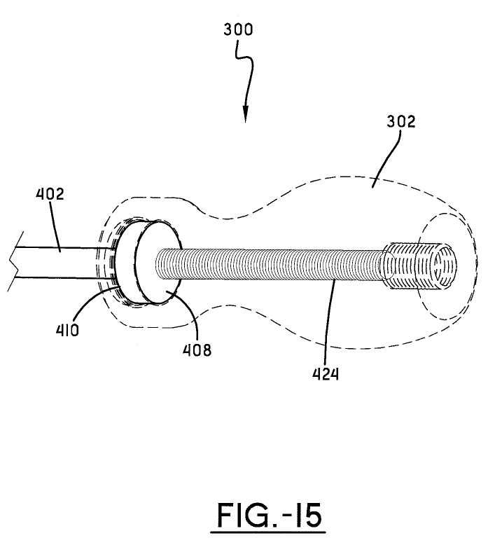

FIG. 16 is a close-up side view of the distal end of an extractor showing the implant in an un-deployed condition.

FIG. 17 is a close-up side view of the distal end of an extractor showing the implant in a deployed condition.

FIG. 18 is a close-up side view of the distal end of an extractor similar to that shown in FIG. 17 but showing a portion of the implant removed.

FIG. 19 is a proximal end view of an extractor according to another embodiment of this invention.

FIG. 20 is a top view of the extractor shown in FIG. 19.

FIG. 21 is a side view of the extractor shown in FIG. 19.

### DETAILED DESCRIPTION OF INVENTION

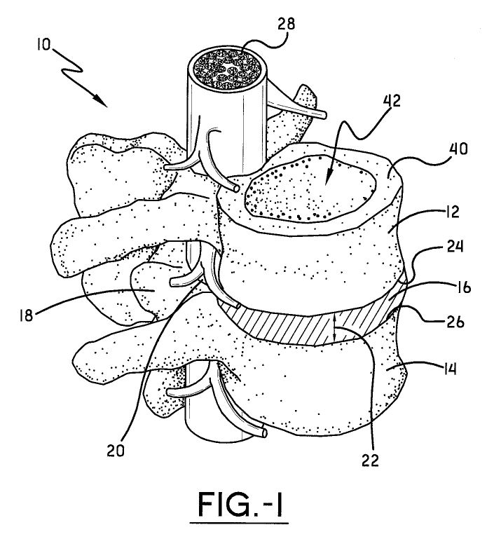

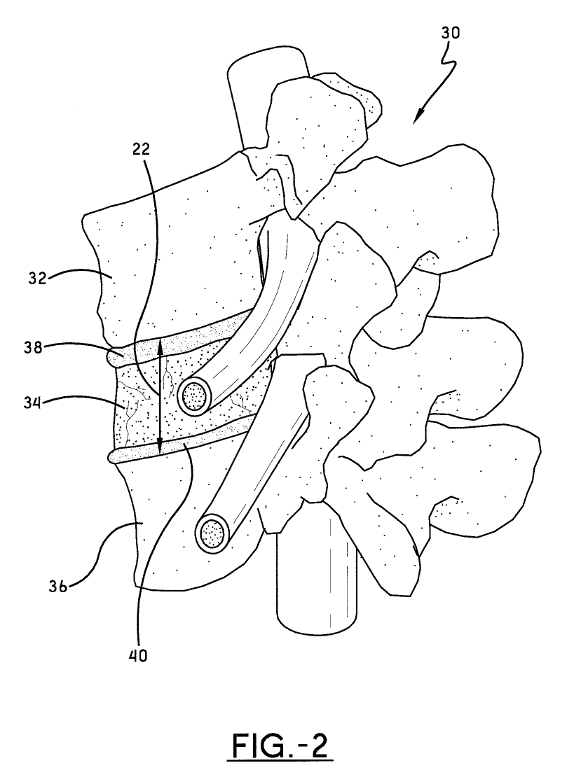

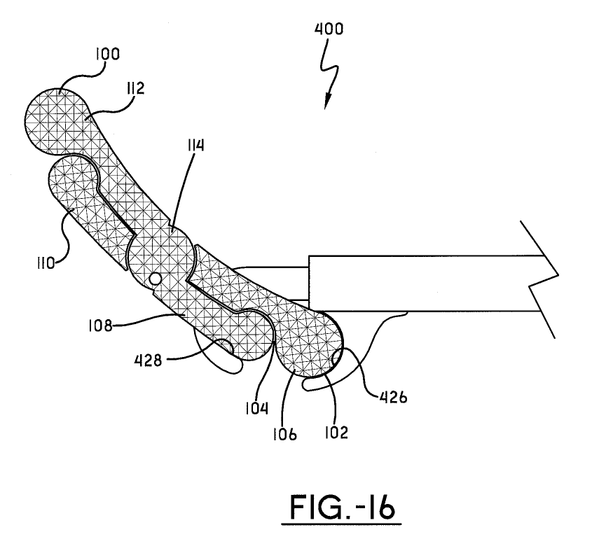

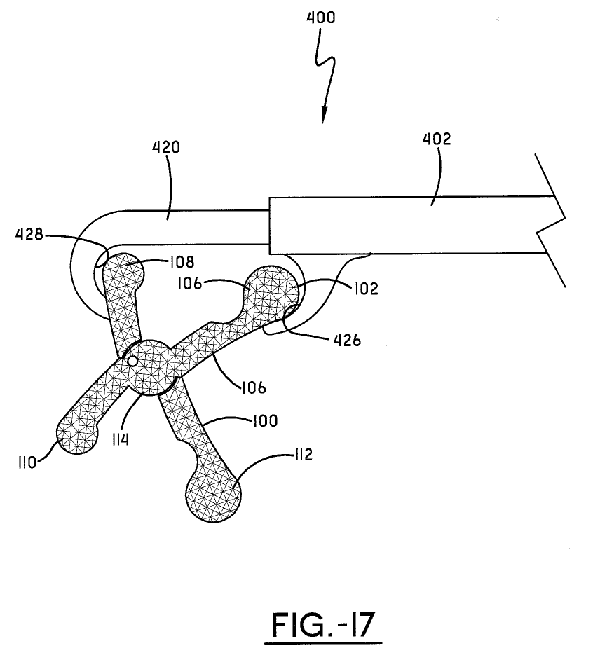

Referring now to the drawings wherein the showings are for purposes of illustrating embodiments of the invention only and not for purposes of limiting the same, the surgical extractor 200, 200a of this invention can be used to extract and, in some embodiments such as shown by comparing FIG. 17 with FIG. 16, un-deploy an implant 100 within a vertebral space 22. By vertebral space it is meant the space in a spinal column where the implant 100 will be placed. In one embodiment, shown in FIG. 1, a spinal segment 10 is made up of two vertebrae 12, 14 attached together by ligaments with a disc 16 separating them. Facet joints 18 fit between the two vertebrae 12, 14 and allow for movement. The neural foramen 20 between the vertebrae 12, 14 allow space for the nerve roots to travel freely from the spinal cord 28 to the body. If it is required to remove the disc 16 and replaced it with an implant 100, the space occupied by the disc 16, the intradiscal space between the two adjacent vertebral bodies 12, 14, defines the vertebral space 22. In another embodiment, shown in FIG. 2, a spinal segment 30 is made up of three vertebrae 32, 34, 36 attached together by ligaments. If it is required to remove the middle vertebra 34 (it is shown diseased) along with the adjacent discs 38, 40, such as may be required because of a corpectomy defect, and replaced them with an implant 100, the space between the two outer vertebral bodies 32, 36, defines the vertebral space 22. It should be understood that these are simply two non-limiting examples of the vertebral space 22 into which an implant 100 can be inserted and then extracted according to this invention because any vertebral space chosen with the sound judgment of a person of skill in the art can be used. As the components and operation of a spinal column is well known to those of skill in the art, further detail will not be provided here.

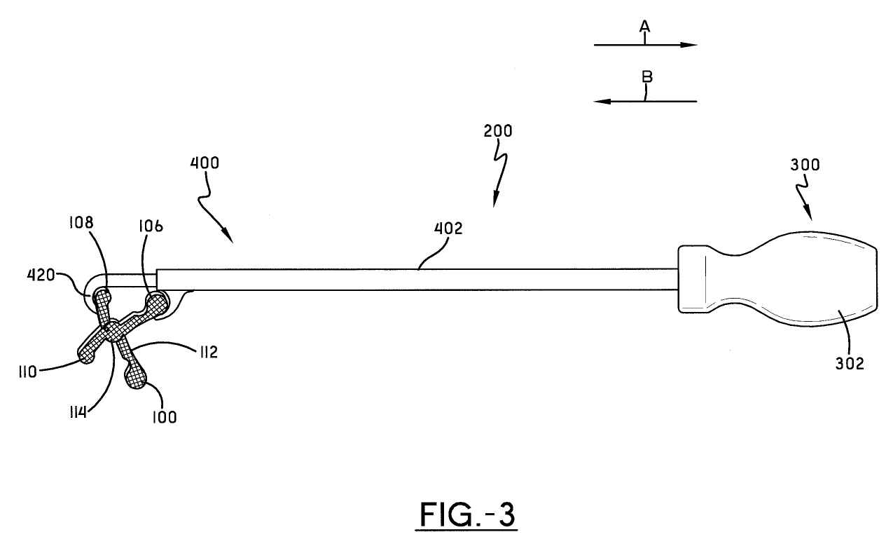

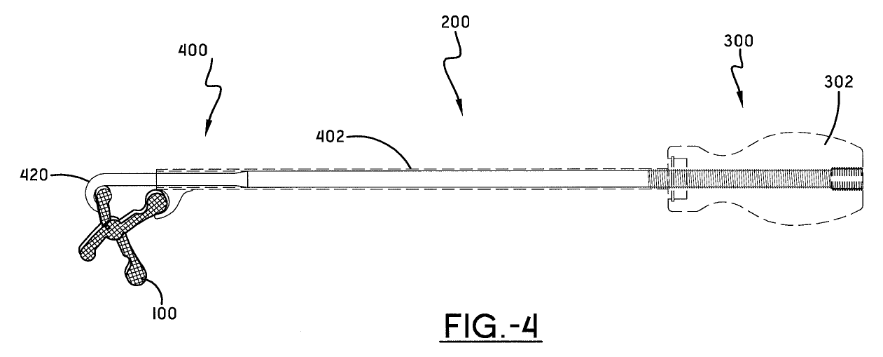

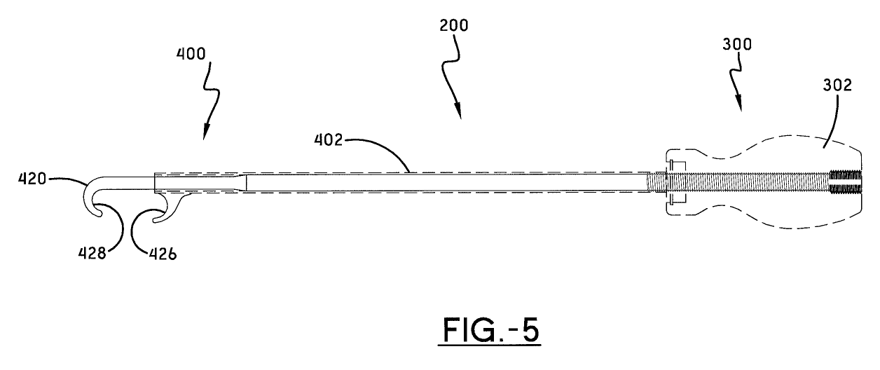

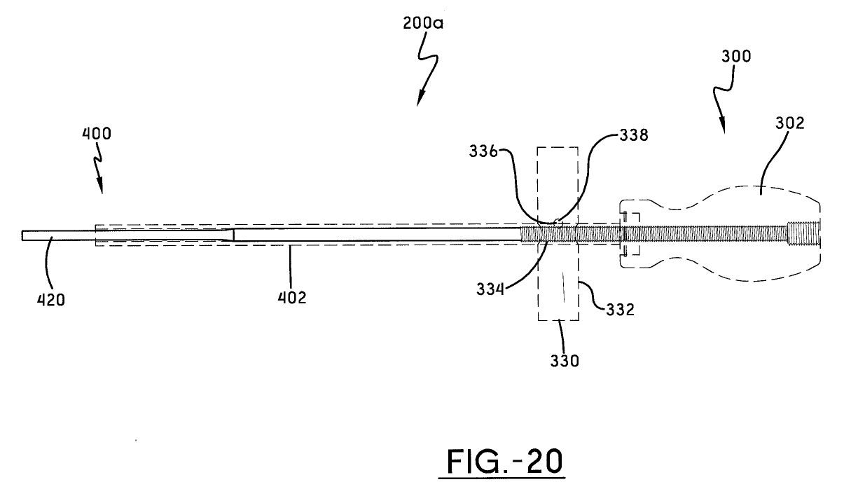

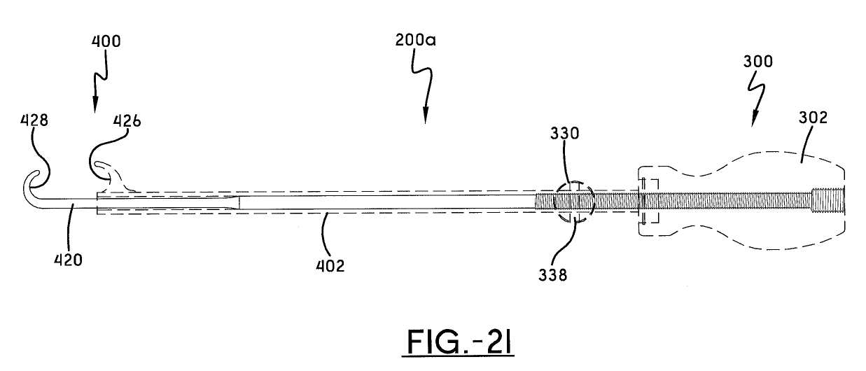

With reference now to FIGS. 3-5 and 19-21, the extractor 200 and 200a of this invention in some embodiments may include a handle mechanism 300 and an implant gripping mechanism 400. Each of these mechanisms will be described in more detail below. Note that throughout this patent the term “proximal” shall refer to direction A as shown in FIG. 3 (toward the handle end of the extractor) and the term “distal” shall refer to direction B as shown in FIG. 3 (toward the implant end of the extractor). These terms are not used to limit this invention in any way but only to provide a direction reference.

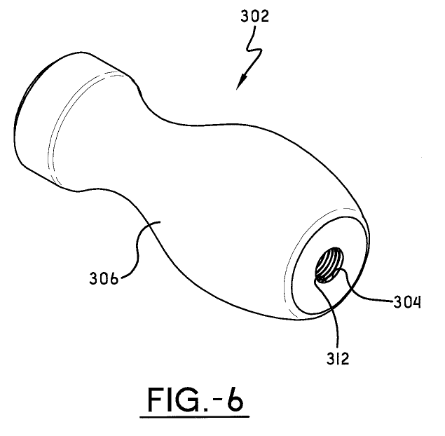

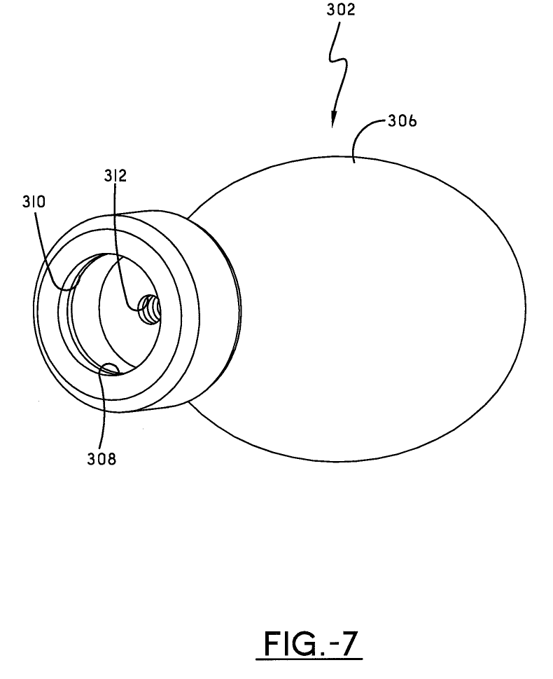

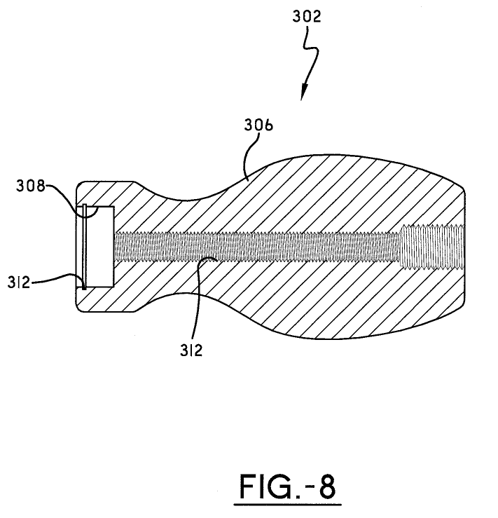

With reference now to FIGS. 3-8 the handle mechanism 300 may include a handle 302 that may be held by the surgeon and used to manipulate the extractor 200 during surgery. The handle 302 may be generally cylindrical and may have an opening 304 throughout its length. This opening 304 can be used for purposes described below and may be positioned substantially in the radial center of the handle 302. The outer surface of the handle 302 may be contoured along the handle length, as shown, and may have a textured region 306 to improve the grip for the surgeon. The handle 302 may be adapted to attach to the implant gripping mechanism 400. In one specific embodiment, the handle 302 may have a cavity 308, a groove 310, and a threaded region 312, defining a portion of the opening 304, for this purpose as well be described in further detail below.

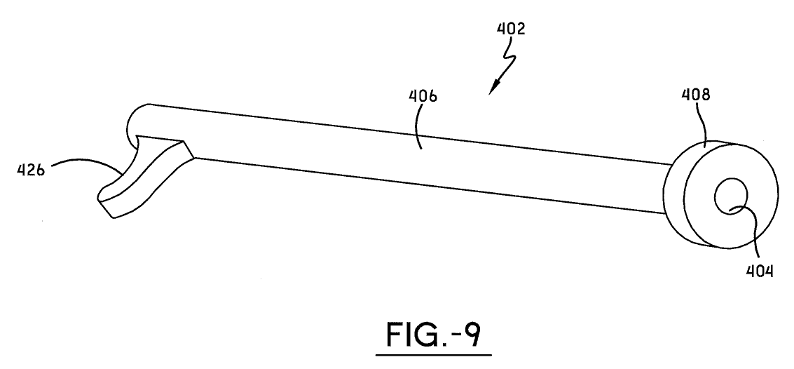

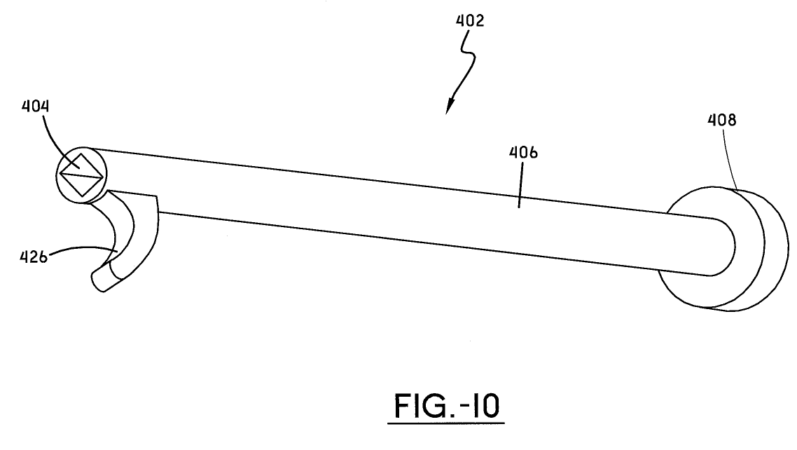

With reference now to FIGS. 1-5 and 9-10, the implant gripping mechanism 400, which is used to grip the implant 100 so that the implant 100 can be removed or extracted from the vertebral space 22, will now be described. The implant gripping mechanism 400 may include a first grip member 402, having a length L1, that may be a tube having have a tube opening 404 throughout its length that can be used for purposes described below and may be positioned substantially in the radial center of the first grip member 402. The first grip member 402 may have a mid-portion 406 with a cross-sectional area. For the embodiment shown, the proximal end has a head 410 with a cross-sectional area that is larger than the cross-sectional area of the mid-portion 406. While both the mid-portion 406 and the head 410 are cylindrically shaped, it should be understood that other shapes will work well with this invention.

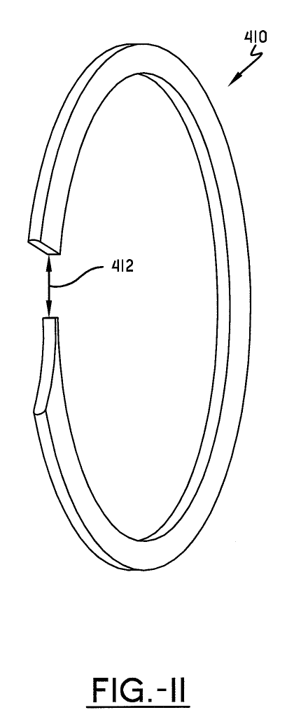

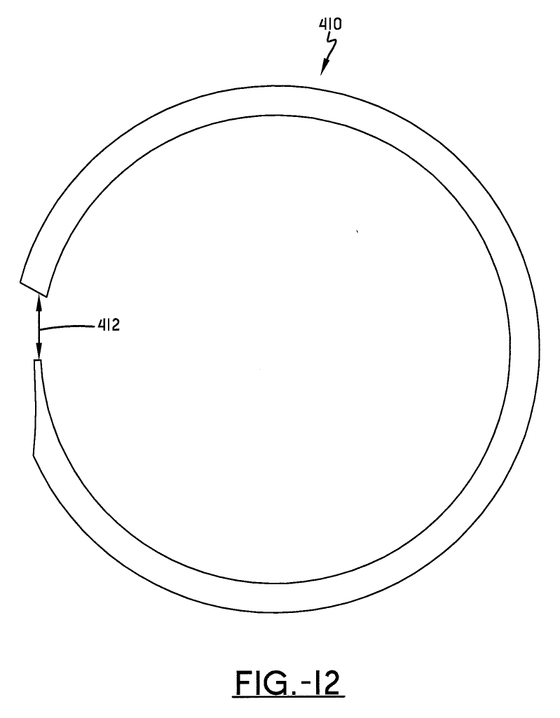

With reference now to FIGS. 1-12, to connect the first grip member 402 to the handle 302, the head 410 is inserted within the cavity 308. A retaining member 410 may then be used to secure the head 410 within the cavity 308. For the embodiment shown, the retaining member 410 is a retaining ring having a gap 412. After the head 410 is positioned within the cavity 308, the retaining ring 410 can be placed around the mid-portion 406 of the first grip member 402. The retaining ring 410 can then be: (1) compressed (narrowing the size of the gap 412); (2) then positioned within the groove 310; and, (3) then released so that the retaining ring 410 expands, in a spring-like fashion, (the size of the gap increases) and at least a portion of the outer edge of the retaining ring 410 contacts the first grip member within the groove 310. If it becomes necessary to disconnect the first grip member 402 from the handle 302, the retaining ring 410 can be compressed and removed from the groove 310. The head 410 can then be easily removed from the cavity 308.

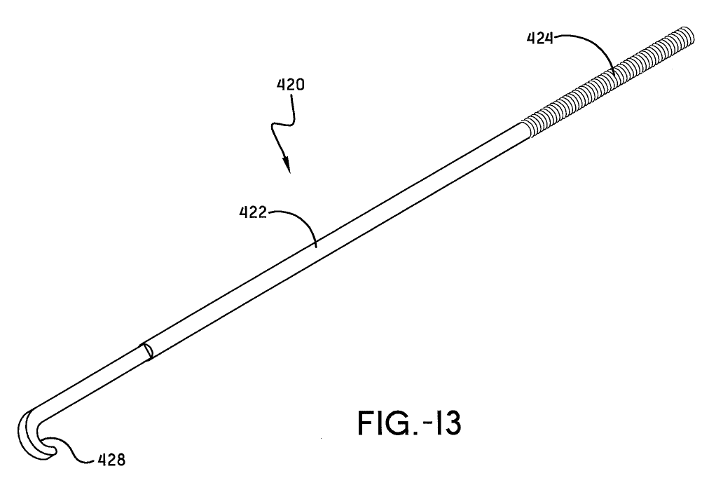

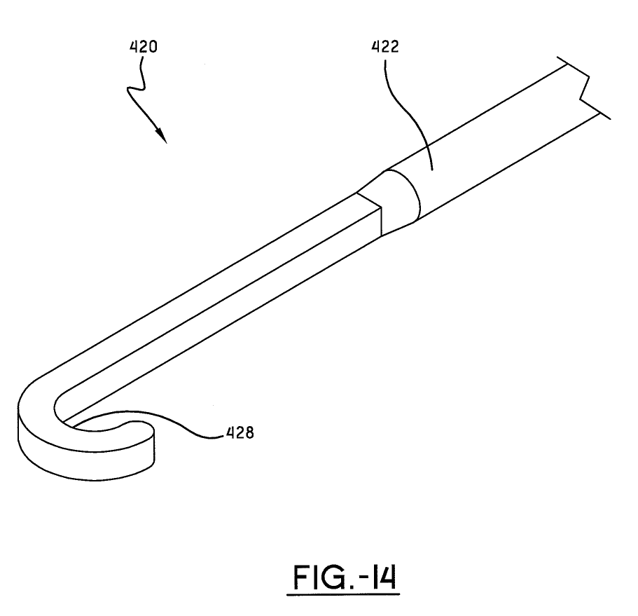

With reference now to FIGS. 1-5 and 13-14, the implant gripping mechanism 400 may also include a second grip member 420 having a length L2 and a mid-portion 422. For the embodiment shown, mid-portion 422 is received within the tube opening 404 of the first grip member 402 and the proximal and distal ends of the second grip member 420 extend out of the proximal and distal ends, respectively, of the first grip member 402. The second grip member 420 can be moved longitudinally within the tube opening 404 in the first grip member 402 as will be described further below. The proximal end of the second grip member 420 may have a threaded region 424 on its outer surface that threadingly engages the threaded region 312 in the handle 302 as will also be described further below.

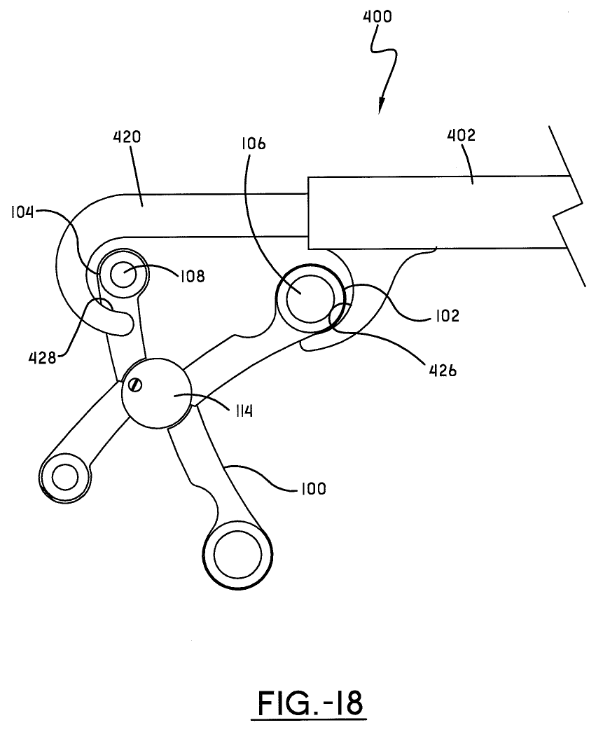

With reference now to FIGS. 1-5, 10, 14 and 16-18, the distal end of the first grip member 402 has a first contact surface 426 that is used to contact a later to be described first engagement surface 102 on the implant 100. Similarly, the distal end of the second grip member 420 has a second contact surface 428 that is used to contact a later to be described second engagement surface 104 on the implant 100. For the embodiment shown, the first and second contact surfaces 426, 428 are formed on portions of the first and second grip members 402, 420, respectively that extend substantially perpendicularly from the longitudinal axes of the first and second grip members 402, 420. In one embodiment, the contact surfaces 426, 428 are shaped to match the shapes of the first and second engagement surfaces 102, 104, respectively. In one specific embodiment, shown, the contact surfaces 426, 428 and the first and second engagement surfaces 102, 104 are curvilinear. In another specific embodiment, not shown, the contact surfaces 426, 428 and the first and second engagement surfaces 102, 104 are linear. In yet another specific embodiment, not shown, the contact surfaces 426, 428 and the first and second engagement surfaces 102, 104 include a pin in slot connection, as is known by those of skill in the art. In one embodiment, shown, the contact surfaces 426, 428 face each other. It should be understood that all the contact surface embodiments just described are exemplary only as any arrangement for the contact surfaces chosen with the sound judgment of a person of skill in the art will work with this invention.

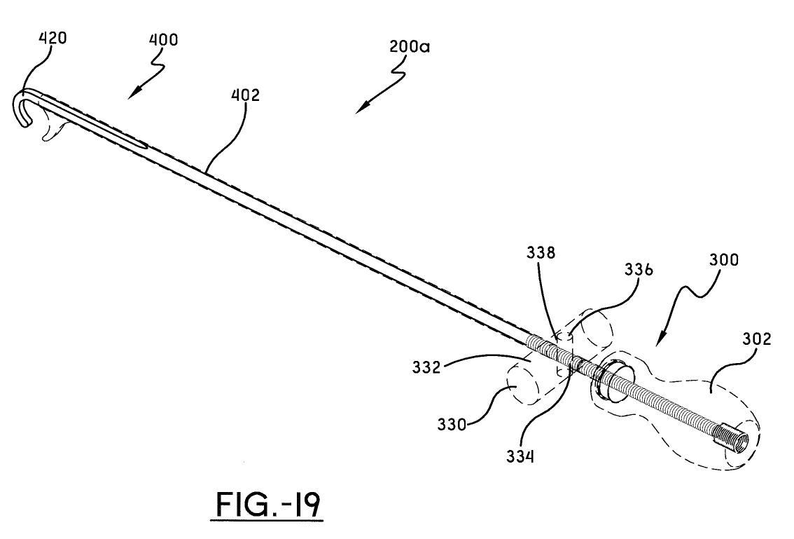

With reference now to FIGS. 19-21, another extractor 200a is shown. Many of the components of this extractor 200a are similar to those described above and given the same reference numbers so they will not be described here. What should be noted, however, is that the handle mechanism 300 also includes a second handle 330 that is used by the surgeon in maneuvering the extractor 200a during surgery. The second handle 330 may be generally cylindrical and may have a textured region 332 to improve the grip for the surgeon. While the second handle 330 can be attached to the extractor 200a in any manner chosen with the skill of a person of skill in the art, for the embodiment shown the second handle 330 is attached to the implant gripping mechanism 400. More specifically, the second handle 330 may have a first opening 334 that receives the first grip member 402 and the second grip member 420 as well as a second opening 336 that receives a dowel pin 338. To secure the second handle 330 to the extractor 200a, the dowel pin 338 is inserted through one end of the second opening 336, through a groove (not shown) formed in an outer surface of the first grip member 402 and then through the opposite end of the second opening 336. In this way, the second handle 330 is secured to the first grip member 402 but permits the second grip member 420 to move relative to the second handle 330.

With reference now to all the FIGURES, the extractors 200, 200a of this invention may be used to remove or extract and, if required, un-deploy any implant chosen with the sound judgment of a person of skill in the art. To understand the meaning of the term “un-deploy,” the term “deploy” will first be described. The term “deploy” as used in this patent refers to any adjustment of an implant after the implant has been initially placed into the vertebral space that involves relative motion of one portion of the implant with respect to another portion of the implant. Non-limiting examples of deployment include implants that have one portion that pivots or moves curvilinearly with respect to another portion and implants that have one portion that slides or moves linearly with respect to another portion. Implants that expand in any manner and in any direction fall under the definition of “deploy.”

The term “un-deploy” as used in this patent refers to any adjustment of a deployed implant within the vertebral space that involves relative motion of one portion of the implant with respect to another portion of the implant. In one embodiment, un-deployment of an implant means returning the implant to its pre-deployed condition. However, it is not necessary for the implant to be returned to its pre-deployed condition for it to be un-deployed according to this patent. It should also be noted that the not all implants are deployable. If this is the case, it should be noted that the extractors 200, 200a of this invention as described above will work well to extract such an implant.

With reference now to FIGS. 3-4 and 16-18, the implant 100 used with the extractors 200, 200a of this invention can be of any type chosen with the sound judgment of a person of skill in the art. The implant may be, for non-limiting examples, any of the implants described in commonly owned U.S. patent application Ser. No. 11/236,068, publication number US 2007/0073398, published on Mar. 29, 2007, titled SPINE SURGERY METHOD AND IMPLANT, which is incorporated herein by reference. While the embodiments discussed below are to implants having four outer posts, it should be understood that other post arrangements are also contemplated. In one embodiment, for example, more than four posts may be used. In another embodiment, less than four posts may be used. In yet another embodiment, one of the posts (or the only post or central support structure) is positioned substantially in the axial center of the implant. In this case it may be desirable to contact the central post for extraction purposes and/or for un-deployment purposes. In any case, two surfaces of the implant, the previously noted first and second engagement surfaces 102, 104, are to be contacted by the extractor as will be explained further below.

With reference again to all the FIGURES, the operation of the extractor 200 of this invention will now be described. First, it should be noted that the implant 100 may have been inserted within the vertebral space 22 in any manner. The implant may be inserted, for non-limiting examples, by any of the insertion techniques and devices described in commonly owned U.S. patent application Ser. No. 11/756,168, titled SPINE SURGERY METHOD AND INSTRUMENTATION and commonly owned U.S. patent application Ser. No. 11/108,625, titled SPINE SURGERY METHOD AND INSERTER, both of which are incorporated herein by reference. In one embodiment, the implant is extracted during the same surgical procedure as when the implant is inserted. This may occur, for some non-limiting examples, when the surgeon discovers that the implant is the wrong size for the patient, that the implant will not deploy properly, or that there are unexpected difficulties related to the patient's spine structure. In another embodiment, the implant is extracted in a separate surgical procedure from the surgical procedure used to insert the implant using any method chosen with the sound judgment of a person of skill in the art. The vetebral space 22 and the implant may be approached, for example, using universally accepted methods for anterolateral, posterior, or posterolateral (transforaminal) discectomy.

With continuing reference to all the FIGURES, once the implant type, style, and size has been determined, the surgeon determines what two surfaces of the implant 100 will be used as the first and second engagement surfaces 102, 104, which are to be gripped by the extractor 200 or 200a. Next, the surgeon assembles the appropriate extractor. It should be noted that the same handle mechanism 300 can be used with numerous implant gripping mechanisms 400. The specific dimensions of the first grip member 402 and the second grip member 420, for example, can be any chosen to properly access and grip the implant to be extracted and may vary, for example, depending on patent parameters (such as patient size) and whether the spinal surgery is done open or via MIS techniques. The surgeon may decide to use a gripping mechanism 400 that has at least one of the contact surfaces 426 or 428 with a shape to match the corresponding at least one of the shapes of the engagement surfaces 102 or 104. For the specific embodiment shown, both of the contact surfaces 426, 428 as well as both of the engagement surfaces 102, 104 are curvilinear. The use of matching surfaces may improve the ability of the implant gripping mechanism 400 to grip the implant 100. As noted above, in another embodiment the connection of the contact surface 426 and/or 428 to the corresponding engagement surface 102 and/or 104 may include a pin in slot connection. However, it should be noted that it is not required that the contact surfaces 426, 428 match the engagement surfaces 102, 104.

Still referring to all the FIGURES, once the surgeon has access to the implant 100 within the vertebral space 22 and is satisfied that the correct extractor 200, or 200a has been assembled, a distractor (not shown) is then placed within the vetebral space 22 and distraction to the selected level of annular tension to remove the implant 100 is achieved. The degree of this distraction would be based on surgeon preference and/or the implant 100 height. With this optimal distraction, further discectomy, or removal of disc material, may be accomplished if required.

With continuing reference to all the FIGURES, the extractor 200 or 200a is then affixed or gripped to the implant 100 using the implant gripping mechanism 400. More specifically, the surgeon first determines the desired space required between the first and second contact surfaces 426, 428 to permit the extractor 200, 200a to grip the implant 100. This desired space between the first and second contact surfaces 426, 428 may be achieved by rotating the handle 302 with respect to the second grip member 420 as described above. If the extractor 200a uses the second handle 330, the surgeon may grip the second handle 330 with one hand while rotating the handle 302 with the other hand. Next, the surgeon maneuvers the extractor 200 or 200a using the handle mechanism 300 into place juxtaposed to the implant 100. The surgeon then rotates the handle 302 with respect to the second grip member 420 to decrease the space between the first and second contact surfaces 426, 428. The surgeon continues this rotation until the first and second contact surfaces 426, 428 are properly in contact with the engagement surfaces 102, 104 of the implant 100. In one specific embodiment, the first engagement surface 102 is on the outer surface of one of the posts 106 on a first portion 110 of the implant 100 and the second engagement surface 104 is on the outer surface of one of the posts 108 on a second portion 112 of the implant 100.

Still referring to all the FIGURES, the next stage of surgery depends on whether the implant 100 needs to be placed into a un-deployed condition before it is to be extracted. If it does, then the surgeon continues the rotation of the handle 302 with respect to the second grip member 420 to decrease further the space between the first and second contact surfaces 426, 428 thereby causing relative motion of one portion of the implant 100 with respect to another portion of the implant 100 to achieve the un-deployed condition. In one specific embodiment, as the space between the first and second contact surfaces 426, 428 is decreased, the first portion 110 of the implant 100 pivots with respect to the second portion 112 about, for example, a pivot point 114.

With continuing reference to all the FIGURES once the implant 100 is in the desired condition (un-deployed or not) to be removed, the surgeon now simply moves the extractor 200 or 200a and thus the implant 100 which is gripped by the extractor using the handle mechanism 300 outside of the vertebral space 22 and then outside of the patient. After the implant 100 is removed from the patient, it may be removed from the extractor 200 or 200a by rotation of the handle 302 with respect to the second grip member 420 to increase the space between the first and second contact surfaces 426, 428 until the implant 100 is released. After the implant 100 is removed, the surgeon can insert a new implant and/or perform any additional surgical techniques that may be required.

Still referring to all the FIGURES, it should be noted that all the extractor embodiments may be formed of any biocompatible material suitable for surgical instruments.

Multiple embodiments have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

Having thus described the invention, it is now claimed:

1. A device comprising: a surgical extractor for use in extracting an implant from a vertebral space, the extractor comprising:
a first handle for use by a surgeon in maneuvering the surgical extractor, the first handle having an opening;
an implant gripping mechanism comprising:
(A) a first grip member having a first end attached to the first handle and a second end comprising a first contact surface; and,
(B) a second grip member having a first end threadingly received within the opening in the first handle and a second end comprising a second contact surface;
wherein the first handle can be rotated in a first direction with respect to the second grip member to cause the first and second contact surfaces to move relatively toward each other to grip the implant; and,
wherein the first handle can be rotated in a second direction with respect to the second grip member to cause the first and second contact surfaces to move relatively away from each other to release the implant.

2. The device of claim 1 wherein the first grip member is a tube with a tube opening along its length that receives the second grip member.

3. The device of claim 1 wherein the first and second contact surfaces are shaped to match the surfaces of the implant which they contact.

4. The device of claim 1 wherein:
the first grip member comprises a mid-portion having a first cross-sectional area;
the first end of the first grip member comprises a head having second cross-sectional area that is substantially larger than the first cross-sectional area;
the first handle has a cavity that receives the head; and,
wherein the device further comprises a retaining member that retains the head within the cavity.

5. The device of claim 4 wherein:
the first handle has a groove defining a portion of the outer surface of the cavity; and,
the retaining member is received within the groove

6. The device of claim 1 further comprising:
a second handle for use by a surgeon in maneuvering the surgical extractor, the second handle being positioned between the first handle and the first contact surface.

7. The device of claim 6 wherein the second handle is supported to the first grip member and has an opening that receives at least a portion of the second grip member.

8. The device of claim 6 wherein:
the first handle has a first longitudinal axis;
the second handle has a second longitudinal axis; and,
when the surgical extractor is assembled, the first longitudinal axis is substantially perpendicular to the second longitudinal axis.

9. The device of claim 1 wherein:
the first grip member has a first longitudinal axis;
the second grip member has a second longitudinal axis;
the first contact surface is formed on a portion of the first grip member that extends substantially perpendicularly from the first longitudinal axis; and,
the second contact surface is formed on a portion of the second grip member that extends substantially perpendicularly from the second longitudinal axis.

10. A surgical system comprising:
an implant comprising:
(A) a first portion having a first engagement surface;
(B) a second portion having a second engagement surface; and,
(C) wherein the implant may be deployed within a vertebral space by moving the second portion with respect to the first portion in a first direction; and,
a surgical extractor for use in un-deploying the implant within the vertebral space and for use in extracting the implant from the vertebral space, the surgical extractor comprising:
(A) a first handle for use by a surgeon in maneuvering the surgical extractor, the first handle having an opening;
(B) an implant gripping mechanism comprising:
(1) a first grip member having a first end attached to the first handle and a second end comprising a first contact surface; and,
(2) a second grip member having a first end threadingly received within the opening in the first handle and a second end comprising a second contact surface;
(C) wherein the first handle can be rotated in a first direction with respect to the second grip member to:
(1) cause the first contact surface to contact the first engagement surface;
(2) cause the second contact surface to contact the second engagement surface;
(3) cause the implant to be un-deployed within the vertebral space by moving the second portion with respect to the first portion in a second direction that is substantially different than the first direction; and,
(4) grip the implant for removal from the vertebral space.

11. The surgical system of claim 10 wherein the first and second contact surfaces are shaped to match the first and second engagement surfaces, respectively.

12. The device of claim 11 wherein the first and second contact surfaces and the first and second engagement surfaces are substantially curvilinear.

13. The device of claim 10 wherein:
the first engagement surface is on a first post; and,
the second engagement surface is on a second post.

14. The device of claim 10 further comprising:
a second handle for use by a surgeon in maneuvering the surgical extractor, the second handle being positioned between the first handle and the first contact surface.

15. A method comprising the steps of:
(A) providing an implant positioned within a vertebral space, the implant having first and second engagement surfaces;
(B) providing a surgical extractor comprising: (1) a first handle for use by a surgeon in maneuvering the surgical extractor, the first handle having an opening; (2) an implant gripping mechanism comprising: (a) a first grip member having a first end attached to the first handle and a second end comprising a first contact surface; and, (b) a second grip member having a first end threadingly received within the opening in the first handle and a second end comprising a second contact surface;
(C) providing access to the vertebral space;
(D) rotating the first handle with respect to the second grip member to cause the first and second contact surfaces to move relatively toward each other;
(E) continue rotating the first handle with respect to the second grip member to cause the first and second contact surfaces to engage the first and second engagement surfaces, respectively, to grip the implant;
(F) moving the surgical extractor and thereby the implant away from the vertebral space.

16. The method of claim 15 wherein step (B) comprises the step of:
providing the first and second surfaces to match the first and second engagement surfaces, respectively.

17. The method of claim 15 wherein:
step (B) comprises the step of: providing the first grip member as a tube with a tube opening along its length that receives the second grip member; and,
step (D) comprises the step of: moving the second grip member with respect to the first grip member within the tube opening.

18. The method of claim 15 wherein:
step (B) comprises the step of: providing the surgical extractor with a second handle for use by a surgeon in maneuvering the surgical extractor; and,
step (D) comprises the step of: gripping the first handle with a first hand and, simultaneously, gripping the second handle with a second hand.

19. The method of claim 15 wherein:
step (A) comprises the step of: providing the implant with: (1) a first portion having the first engagement surface; (2) a second portion having the second engagement surface; and, (3) wherein the implant is deployed within the vertebral space by the movement of the second portion with respect to the first portion in a first direction; and,
after step (E) but before step (F), the method comprises the step of: continue rotating the first handle with respect to the second grip member to cause the un-deployment of the implant within the vertebral space by causing the movement of the second portion with respect to the first portion in a second direction that is substantially different than the first direction.

20. The method of claim 19 wherein the step of, continue rotating the first handle with respect to the second grip member to cause the un-deployment of the implant within the vertebral space by causing the movement of the second portion with respect to the first portion in a second direction, comprises the step of:
rotating the second portion of the implant with respect to the first portion of the implant in the second direction which is substantially opposite to the first direction.
